(19) United States
(12) Patent Application Publication
(10) Pub. No.: US 2012/0078149 A1
(43) Pub. Date: Mar. 29, 2012

(54) Orthopedic Posture Brace

(76) Inventor: Mahnaz Azimzadeh, Tarzana, CA (US)

(21) Appl. No.: 13/239,089

(22) Filed: Sep. 21, 2011

Related U.S. Application Data
(60) Provisional application No. 61/385,841, filed on Sep. 23, 2010.

(51) Int. Cl. A61F 5/00; B23P 11/00

(52) U.S. Cl. 602/19; 29/428

(57) ABSTRACT

One embodiment of an orthopedic posture brace comprising a holster strap that forms two arm loops when attached to an axial strap and two abdominal straps. When worn, the holster straps encircle the user's shoulders. The axial strap will rest against the thoracic region of the user's back. After the two abdominal straps pass through a loop at the bottom of the axial strap, the two ends not connected to the holster strap may be fastened together around the front of the user's abdomen. Tightening the ends of the abdominal strap will cause tension in the holster straps urging the user's shoulders rearward to encourage proper spinal alignment and reduced slouching or hunching over. The amount of tension generated by tightening the abdominal straps will determine how far back toward the spinal column the user's shoulders are pulled.

### CROSS-REFERENCE TO RELATED APPLICATIONS

The present application claims priority to U.S. Provisional Patent Application Ser. No. 61/385,841 entitled ORTHOPEDIC POSTURE BRACE, filed on Sep. 23, 2010, which is hereby incorporated by reference as to its entirety.

### BACKGROUND

The following is a tabulation of some prior art that presently appears relevant:

U.S. Pat. Nos.

| Pat. No. | Kind Code | Issue Date | Patentee |
|---|---|---|---|
| 756,237 | A | Apr. 5, 1904 | Hunkins |
| 3,116,735 | A | Jan. 2, 1964 | Geimer |
| 3,548,818 | A | Dec. 22, 1970 | Kaplan |
| 4,080,962 | A | Mar. 28, 1978 | Berkeley |
| 6,440,094 | B1 | Aug. 27, 2002 | Maas |
| 7,134,969 | B2 | Nov. 14, 2006 | Citron |
| 7,901,371 | B1 | Mar. 8, 2011 | Yayntraub |

### U.S. Patent Application Publications

| | | | |
|---|---|---|---|
| Publication Number | Kind Code | Publ. Date | Applicant |

None found

### Foreign Patent Document

None found

### Nonpatent Literature Documents

None found

### NOTICE OF COPYRIGHT AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### DISCUSSION OF THE RELATED ART

The natural position of the trunk and shoulders is an erect one, more or less perpendicular to the surface upon which a person is standing, in contrast to a slouched or forward leaning position. Whenever an individual is unable to maintain such posture on their own, their doctor may recommend the use of a posture brace.

All presently known orthopedic posture braces are unnecessarily large making them difficult or impossible to be worn underneath the user's clothing. Furthermore, many of these devices tend to unduly restrict or completely prohibit the movement of the user's shoulders making them extremely uncomfortable to wear for more than a short period of time.

There are a number of posture braces available which are designed to place tension on the user's shoulders and draw them back necessitating erect posture. These braces are large, cumbersome, and force the user to wear the brace over their clothing. Some of these devices are comprised of wide straps which constrict movement causing discomfort to the user.

Furthermore, many of these devices contain large constricting chest bands and the rhomboid portion of these devices are seldom adjustable vertically. The bands that comprise these devices tend to be unnecessarily wide and create bulk when worn. The end result is an uncomfortable brace which causes tension on the shoulders and strain on other parts of the body.

Finally, many of the presently known orthopedic posture braces lack adjustable arms bands or holster straps that would enable the user to quickly and easily adjust the amount of tension created by the brace. This makes the known braces more uncomfortable and less likely to be worn as directed by medical professionals.

A more comfortable back brace which may be hidden underneath one's clothing and is adjustable to fit the contours of each individual's body is a desirable tool for improving posture and health.

Even though several types of posture braces have been proposed, all of the systems heretofore known suffer from a number of disadvantages:
(a) the known braces are large and cumbersome, making it impractical for the user to wear them underneath their clothing;
(b) the known braces unduly limit shoulder movement;
(c) the known braces contain large or constricting chest bands;
(d) the known braces contain large or constricting shoulder straps;
(e) the known braces do not contain vertically adjustable straps in the rhomboid region of the brace;
(f) the known braces cause discomfort making prolonged use impractical or impossible;
(g) the known braces are not adjustable using Velcro or buckles;
(h) the known braces are not designed to accommodate wearers with different heights; and
(i) the known braces do not utilize the healing properties of magnets.

Therefore, there is a need for an orthopedic posture brace which can engage the shoulders, back and chest to facilitate proper posture while being small enough to be hardly visible when worn underneath cloths. Such a brace would also be comfortable enough to be worn an entire day without being taken off because it would not unduly prohibit the user's shoulder and back movement. The present invention effectuates these needs.

### SUMMARY

In accordance with one embodiment, an orthopedic brace that dynamically enhances proper posture and can be worn underneath one's clothing is envisioned. Said back brace has a height adjustable axial strap and adjustable holster strap for maximum user comfort and mobility. One embodiment of the present invention includes an elastomeric axial strap that, when worn, rests against the rhomboid section of the user's back. In one embodiment, the axial strap may be vertically adjustable to accommodate users of different heights.

It is envisioned that in one embodiment the posture brace may have a pair of shoulder-engaging abdomen-encircling straps. These straps would be connected to the axial strap and allow the user to adjust the tension created by the posture brace. Pulling the ends of the abdominal straps located on the front of the brace will lead to an increase in the amount of tension felt by the user.

An orthopedic posture brace having an elastomeric rhomboid section which is adjustable vertically and a pair of shoulder engaging holster straps is envisioned. The holster straps may be loosened or tightened by lengthening or shortening a pair of abdomen encircling straps. Such a brace may be comprised of four pieces. A holster strap which, when worn, runs along the upper back and encircles the user's shoulders; an elastomeric axial portion that runs parallel to the spinal column; and two narrow straps that run underneath each arm, encircle the waste, and join one another in the front of the user's abdomen.

The elastomeric portion of the brace has two ends. One end is attached to the center of the holster strap while the other end forms a loop. In one embodiment, these two pieces may be attached by sewing them together. When worn, the elastomeric portion will lie against the upper back just below the neck of the wearer.

The holster strap contains two ends. Each end is attached to a narrow strap that, when worn, will run underneath the user's arm, through a loop in the elastomeric portion, to the front of the wearer. In one embodiment, the holster strap may be attached to the narrow straps by sewing the ends together. In one embodiment of the holster strap, it may contain two magnets. Each magnet will be located inside the padded piece of the brace resting over the user's upper trapezious muscles. In yet another embodiment, the holster strap may be padded.

The height of the elastomeric portion may be lengthened or shortened. This adjustment may be achieved in a variety of different ways. In one embodiment, the loop end contains a piece of Velcro. The elastomeric portion may be shortened by fastening this end closer to the opposing end. Conversely, it may be lengthened by fastening it further away from the opposing end.

The width of the elastomeric portion is not adjustable. However, it is envisioned that the orthopedic posture brace may be made with different width elastomeric portions in order to enhance the comfort of the user. In one embodiment, the elastomeric portion may be wide to better suit overweight or taller users while in another embodiment it may be narrower to suit thinner users.

As discussed, each end of the holster strap will be connected to a narrow strap. This narrow abdomen-encircling strap will travel underneath the user's arm and into the elastomeric loop on the back of the posture brace. The narrow strap will emerge from the loop of the elastomeric portion on the side of the opposite arm and then travel to the front of the user's abdomen. The two narrow straps on either side of the user will come together in front of his or her abdomen. In one embodiment, the two narrow pieces may be fastened together using a buckle. In yet another embodiment, the two narrow pieces may be joined together using Velcro.

The narrow strap may be constructed from different materials in order to increase user comfort. In one embodiment, the narrow strap may be constructed from an elastomeric fabric while in another the fabric may not stretch at all.

Together, the holster strap and the narrow strap define the shoulder-engaging region through which the arms extend. This region encircles the shoulders and pulls them back to the desired position in a comfortable way. The shoulder-engaging region may be adjusted to better fit the wearer. This adjustment is accomplished by tightening the ends the narrow straps which come together at the front of the user's abdomen.

### ADVANTAGES

Thus several advantages of one or more aspects are to provide:
(a) a device which promotes proper posture by tapping into the user's proprioception for the perception stimuli which is related posture;
(b) a device which promotes proper posture by engaging the user's cerebellum;
(c) a device which promotes proper posture while being small enough to be unnoticeable while worn under a shirt or blouse;
(d) a device which promotes posture and utilizes the healing properties of magnets;
(e) a posture promoting device which reduces nerve pressure and the incidence of arthritis in it's users by making sure that disk height remains in tact;
(f) a posture promoting device which is light, easy to use and comfortable; and
(g) a posture promoting device which can be used by anyone over the age of 12.

These and other advantages of one or more aspects will become apparent from consideration of the ensuing description and accompanying drawings. Although the description above contains many specificities, these should not be construed as limiting the scope of the embodiments but as merely providing illustrations of some of several embodiments. For example, the holster straps may be constructed with magnets in one embodiment and without magnets in another. Magnets have known healing properties and when placed in the holster straps to rest over the user's upper trapezious muscles, magnets can increase oxygenation, tissue healing, and blood flow. Thus the scope of the embodiments should be determined by the claims that are appended and their legal equivalents, rather than by the examples given.

The description of the invention which follows, together with the accompanying drawings should not be construed as limiting the invention to the example shown and described, because those skilled in the art to which this invention pertains will be able to devise other forms thereof within the ambit of the appended claims.

### DESCRIPTION OF THE DRAWINGS

### Figures

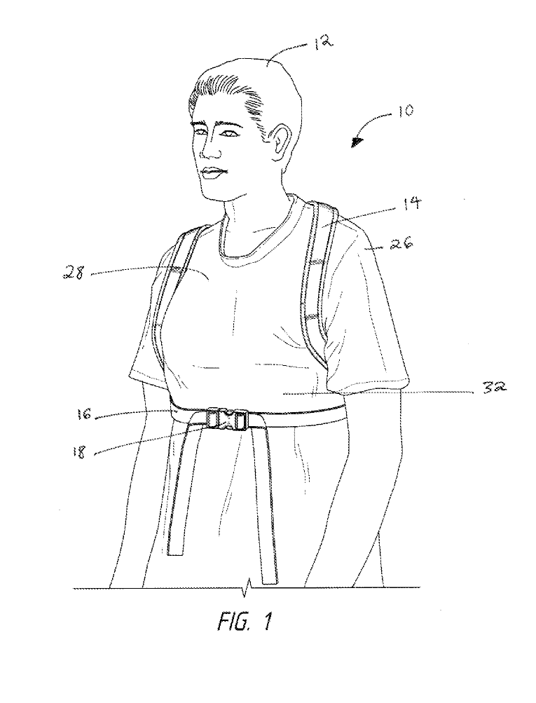

FIG. 1 is a perspective view of the brace illustrated as worn by a user;

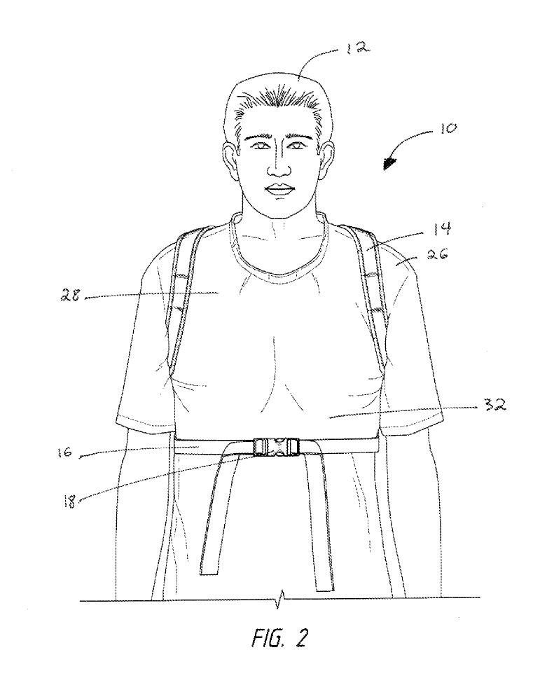

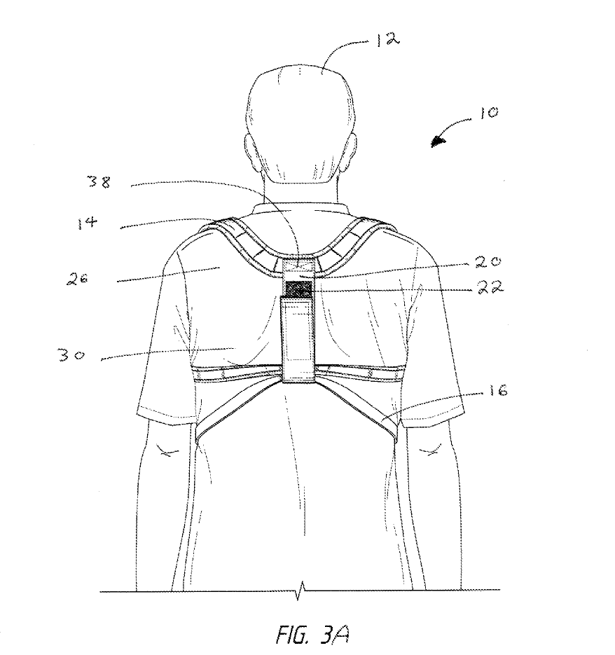

FIG. 2 is a front view of the brace illustrated as worn by a user;

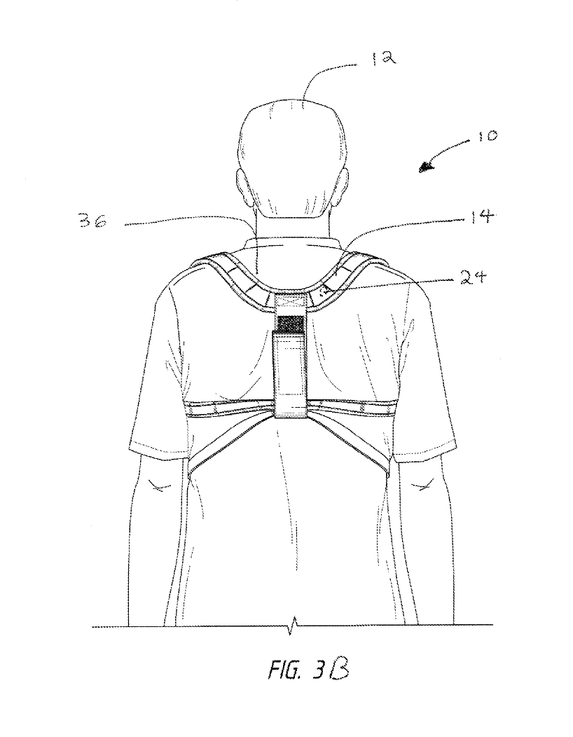

FIG. 3A is a rear view of the brace illustrated as worn by a user;

FIG. 3B is a rear view of the brace with holster strap magnets illustrated as worn by a user;

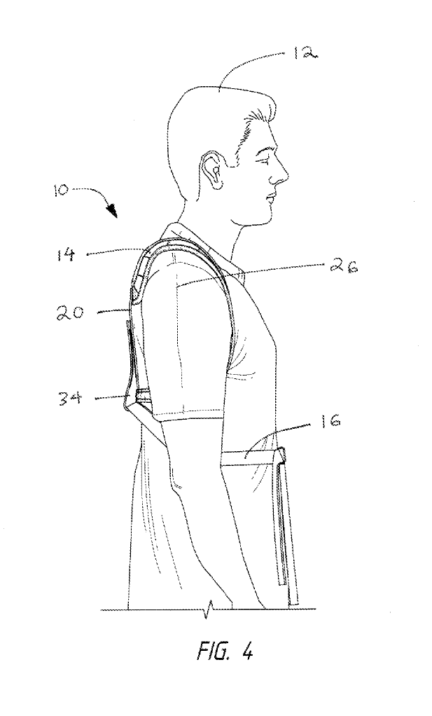

FIG. 4 is a right perspective view of the brace illustrated as worn by a user;

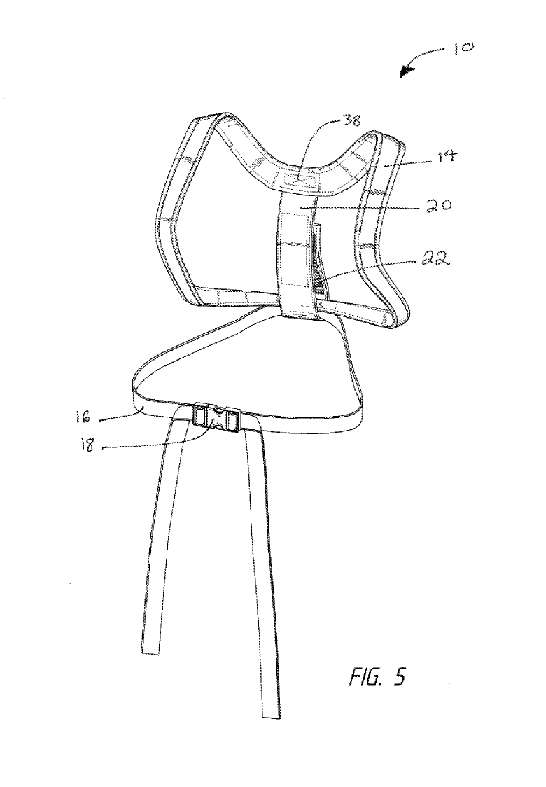

FIG. 5 is a front perspective view of the brace, illustrating the axial strap, the holster straps, and abdominal straps fastened with a buckle;

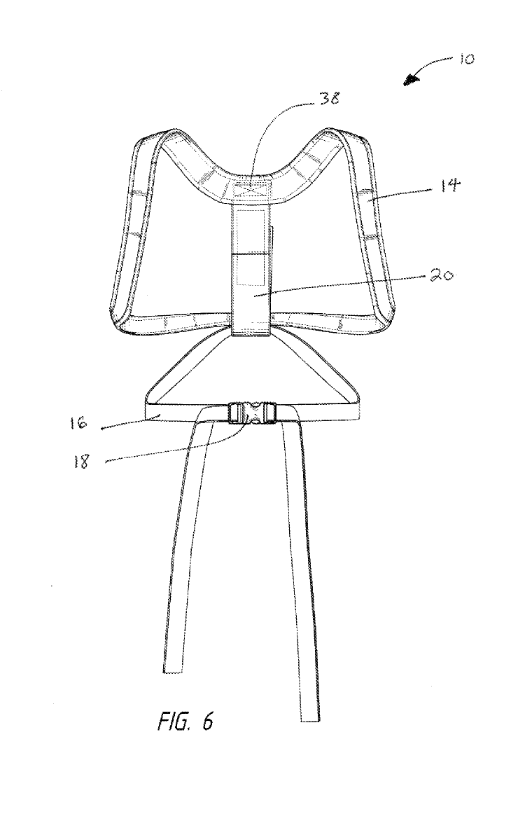

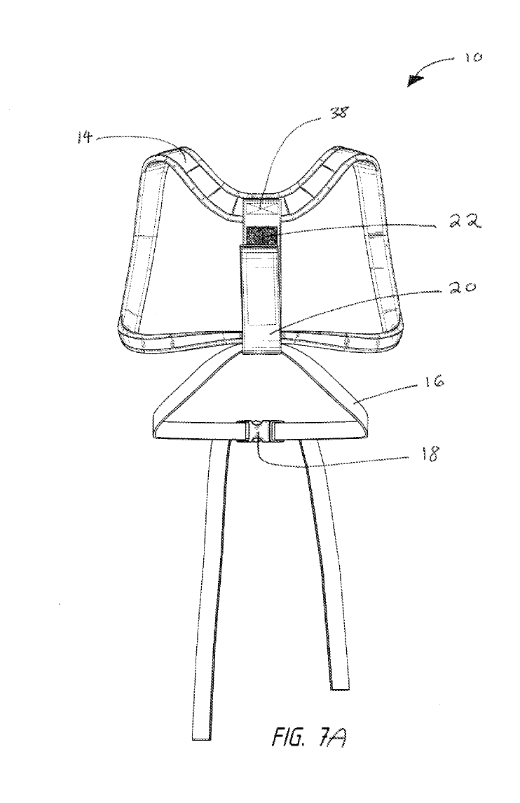

FIG. 6 is a front view of the brace, illustrating the axial strap, the holster straps, and abdominal straps fastened with a buckle;

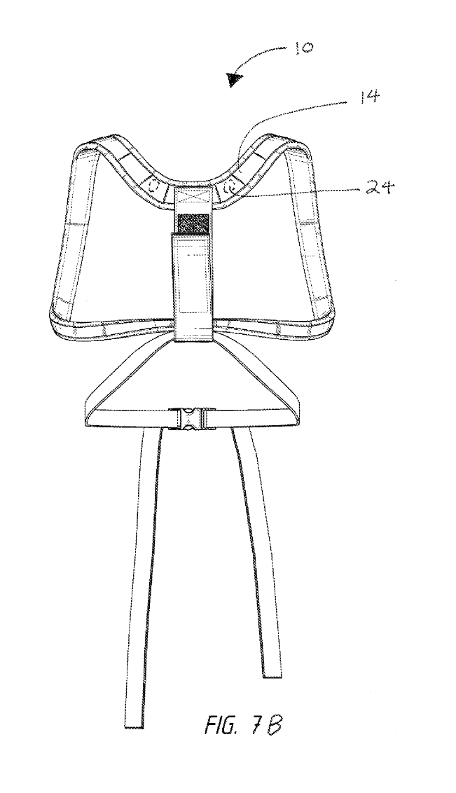

FIG. 7A is a rear view of the brace, illustrating the adjustable Velcro axial strap, the holster straps, and abdominal straps fastened with a buckle;

FIG. 7B is a rear view of the brace, illustrating the adjustable Velcro axial strap, the holster straps with magnets, and abdominal straps fastened with a buckle; and

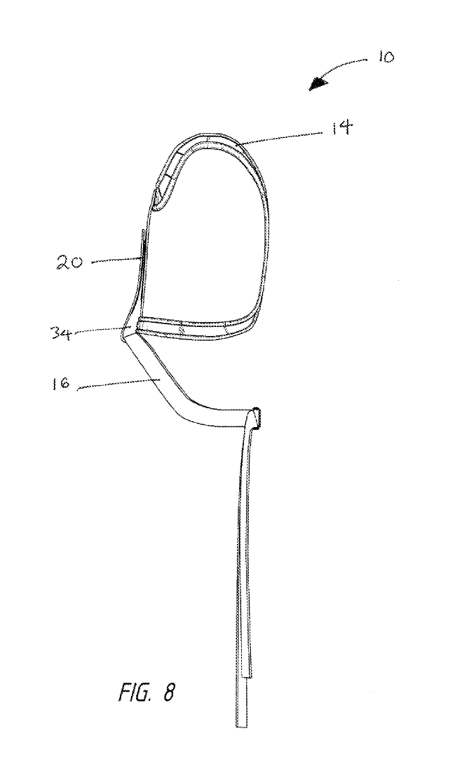

FIG. 8 is a right perspective view of the brace, illustrating the axial strap, the holster straps and abdominal straps fastened with a buckle.

Reference Numerals

| | | | |
|---|---|---|---|
| 10 | posture brace | 12 | user |
| 14 | holster strap | 16 | abdominal strap |
| 18 | buckle | 20 | axial strap |
| 22 | Velcro | 24 | magnet |
| 26 | user's shoulder | 28 | user's chest |
| 30 | user's shoulder blades | 32 | user's abdomen |
| 34 | axial strap loop | 36 | user's upper trapezious |
| 38 | sown portion of axial strap | | |

### DETAILED DESCRIPTION

Illustrative embodiments of the invention are described below. The following explanation provides specific details for a thorough understanding of an enabling description for these embodiments. One skilled in the art will understand that the invention may be practiced without such details. In other instances, well-known structures and functions have not been shown or described in detail to avoid unnecessarily obscuring the description of the embodiments.

One embodiment of the orthopedic posture brace 10 is illustrated in FIGS. 5, 6, 7A, 7B, and 8. When worn correctly as illustrated in FIGS. 1, 2, 3A, 3B, and 4, the posture brace 10 will promote proper posture in the user 12.

The posture brace 10 has two holster straps 14 that, when worn, completely encircle the users shoulders 26. In one embodiment, the holster straps 14 may be padded to provide additional comfort to the user 12. As illustrated in FIG. 3A, the holster straps 14 may be made from it a single piece of material which is divided in two by the axial strap 20 to form two arm loops. They run along the front of the user's chest 28 (FIG. 2) and beneath the user's under arms (FIG. 3A).

The end of each holster strap 14 becomes the beginning of the abdominal strap 16. The abdominal straps 16 and the holster strap 14 may be constructed from a single piece of material or from separate pieces as in the padded holster strap 14 embodiment shown in the figures. As illustrated in FIG. 3A, when the posture brace 10 is worn properly, the holster strap 14 encircles the user's shoulder 26 passes beneath the left under arm, underneath the left shoulder blade 30, through the axial strap loop 34 (FIG. 4) emerging on the opposite side of the user 12 as the an abdominal strap 16.

In one embodiment of a posture brace 10, the two lose ends of the abdominal strap 16 may be fastened around the user's abdomen 32 (FIG. 2) using a buckle 18. In this embodiment, each of the two lose ends of the abdominal strap 16 will contain either the male or female buckle member. Once the buckle 18 is fastened, it can be adjusted to fit securely around the user's abdomen 32 by tugging at the two lose ends of the abdominal strap 16. Tugging on either one of these ends will also tighten the opposing arm loop made by that side of the holster strap 14.

Tightening the ends of the abdominal strap 16 will cause tension in the holster straps 14 urging the user's shoulders 26 rearward to encourage proper spinal alignment and reduce slouching or hunching over. The amount of tension generated by tightening the abdominal straps 16 will determine how far back toward the spinal column the user's shoulders 26 are pulled.

When the user 12 is exhibiting proper posture, the tension on the back will decrease therein. Alternatively, slouching will lead to increased tension. This will in turn tap into the user's proprioception responsible for the perception stimuli related to proper posture and the user's cerebellum reminding the user 12 to stand straight.

For the sake of illustration, only the embodiment where a buckle 18 is used to fasten the abdominal straps 16 is shown in the drawings. Alternately, it is envisioned that the posture brace 10 may utilize or other similar means of securing the two waist straps 16 to one another around the user's abdomen 32 (e.g. a clasp, a belt or Velcro).

The axial strap 20 is situated on the back of the user 12 (FIG. 3A) when worn running along the rhomboid section of the user's back (or in alignment with the spinal column from the top thoracic to the middle the thoracic vertebrae). The axial strap 20 too may be made from a single piece of material. The top end of the axial strap 20 may be connected to the holster straps 14 by sowing the two pieces together. It is envisioned that other connecting means may be used to secure the top of the axial strap 20 to the holster strap 14 (e.g. hot glue). Located beneath the sown portion 38 (FIG. 3A), is a section of Velcro 22 (or other form of hook-and-loop fastener). The Velcro 22 is adhered to the two opposing surfaces of the axial strap 20 to be fastened.

When the two opposing surfaces of the axial strap 20 are fasted, they form the axial loop 34 (FIG. 4) through which the two abdominal straps 16 criss-cross to end up on the opposite end of the user's 12 back. The axial strap 20 and therefore the height of the axial loop 34 can be adjusted by fastening the Velcro 22 higher or lower.

In one embodiment, the axial strap 20 may be made from an elastomeric material.

In another embodiment of the posture brace 10, the holster straps 14 may each contain a magnet 24 (FIG. 3B). Magnets are known to have healing properties including but not limited to, increased oxygenation of the blood, and increasing blood flow which can rejuvenate tissue throughout the body. Each magnet 24 can be situated in the holster strap 14 (FIG. 7B). As illustrated in FIG. 3B, when the posture brace 10 is being worn, the magnets will rest in the upper trapezious 36 region of the user's back.

### CONCLUSIONS, RAMIFICATIONS, AND SCOPE

While a particular form of the invention has been illustrated and described, it will be apparent that various modifications can be made without departing from the spirit and scope of the invention. For example, the waist strap fastening means may be implemented by a variety of different devices. Accordingly, is not intended that the invention be limited, except as by the appended claims.

The teachings provided herein can be applied to other systems, not necessarily the system described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Particular terminology used when describing certain features or aspects of the invention should not be taken to imply that the terminology is being refined herein to be restricted to any specific characteristics, features, or aspects of the posture brace with which that terminology is associated. In general, the terms used in the following claims should not be constructed to limit the posture brace to the specific embodiments disclosed in the specification, unless the above Detailed Description section explicitly define such terms. Accordingly, the actual scope encompasses not only the disclosed embodiments, but also all equivalent ways of practicing or implementing the disclosed posture brace. The above detailed description of embodiments of the posture brace is not intended to be exhaustive or limited to the precise form disclosed above or to a particular field of usage. While specific embodiments of, and examples for, the posture brace are described above for illustrative purposes, various equivalent modifications are possible which those skilled in the relevant art will recognize.

While certain aspects of the posture brace are presented below in particular claim forms, the inventor contemplates the various aspects of the posture brace in any number of claim forms. Accordingly, the inventor reserves the right to add additional claims after filing the application to pursue such additional claim forms for other aspects of the posture brace.

What is claimed is:

1. An orthopedic posture brace comprising:
an axial strap having a top and a bottom to be disposed in alignment with the spinal column from the top thoracic to the middle the thoracic vertebrae when worn;
a holster strap with a right side and a left side extending laterally outward from said axial strap; and
a first and second abdominal strap extending laterally outward in opposite directions from the bottom of said axial strap.

2. The orthopedic posture brace of claim 1 wherein said holster strap contains a magnets situated in the part of said straps worn over the trapezious area of the user's back.

3. The orthopedic posture brace of claim 1 wherein said holster strap is padded.

4. The orthopedic posture brace of claim 1 wherein said first and second abdominal straps are fastened together using a buckle.

5. The orthopedic posture brace of claim 1 wherein said first and second abdominal straps are fastened together using Velcro.

6. An orthopedic posture brace comprising:
an elastomeric axial strap having a top and a bottom to be disposed in alignment with the spinal column from the top thoracic to the middle the thoracic vertebrae when worn;
a holster strap having a right and left side extending laterally outward from said axial strap in opposite directions, wherein said strap contains at least two magnets; and
a first and second abdominal strap extending laterally outward in opposite directions from the bottom of said axial strap.

7. The orthopedic posture brace of claim 6 wherein said holster strap is padded.

8. The orthopedic posture brace of claim 6 wherein said abdominal straps may be fastened using a buckle.

9. The orthopedic posture brace of claim 6 wherein the height of said elastomeric axial strap may be adjusted using Velcro.

10. A method of constructing an orthopedic posture brace comprising:
(a) providing an axial strap, a holster strap having a first and second end, and a first and second abdominal strap;
(b) connecting said axial strap to the middle of said holster strap;
(c) connecting one end of said first abdominal strap to the first end of said holster strap; and
(d) connecting one end of said second abdominal strap to the second end of said holster strap.

11. The method of claim 10 wherein said holster straps are padded.

12. The method of claim 10 wherein the height of said axial strap is adjustable using Velcro.

13. The method of claim 10 wherein the first and second abdominal straps may be fastened to one another using a buckle.

14. The method of claim 10 wherein the first and second abdominal strap may be fastened to one another using Velcro.
